(19) United States
(12) Patent Application Publication
(10) Pub. No.: US 2022/0092800 A1
(43) Pub. Date: Mar. 24, 2022

(54) REAL-TIME TRACKING FOR FUSING ULTRASOUND IMAGERY AND X-RAY IMAGERY

(71) Applicant: KONINKLIJKE PHILIPS N.V., EINDHOVEN (NL)

(72) Inventors: Grzegorz Andrzej TOPOREK, BOSTON, MA (US); Marcin Arkadiusz BALICKI, CAMBRIDGE, MA (US)

(21) Appl. No.: 17/421,783

(22) PCT Filed: Jan. 13, 2020
(86) PCT No.: PCT/EP2020/050624

§ 371 Date: Jul. 9, 2021

Related U.S. Application Data
(60) Provisional application No. 62/792,451, filed on Jan. 15, 2019.

(51) Int. Cl. G06T 7/33; A61B 6/04; A61B 6/08; A61B 6/00; A61B 90/00; A61B 8/08

(52) U.S. Cl. CPC G06T 7/337; G06T 2207/20221; A61B 6/08; A61B 6/487; A61B 6/5247; A61B 6/582; A61B 90/39; A61B 6/4441; A61B 6/54; A61B 8/5261; A61B 2090/3966; G06T 2207/10116; G06T 2207/10064; G06T 2207/10132; G06T 2207/30204; A61B 6/0492

(57) ABSTRACT

A registration system includes a controller (160). The controller (160) includes a memory (162) that stores instructions; and a processor (161) that executes the instructions. When executed, the instructions cause the controller (160) to execute a process that includes obtaining a fluoroscopic X-ray image (S810) from an X-ray imaging system (190), and a visual image (S820) of a hybrid marker (110) affixed to the X-ray imaging system (190) from a camera system (140). A transformation between the hybrid marker (110) and the X-ray imaging system (190) is estimated (S830) based on the fluoroscopic X-ray image. A transformation between the hybrid marker (110) and the camera system (140) is estimated (S840) based on the visual image. Ultrasound images from an ultrasound system (156) are registered (S850) to the fluoroscopic X-ray image from the X-ray imaging system (190) based on the transformation estimated between the hybrid marker (110) and the X-ray imaging system (190), so as to provide a fusion of the ultrasound images to the fluoroscopic X-ray image.

### BACKGROUND

Procedures in the field of structural heart disease are increasingly becoming less invasive. For example, transcatheter aortic valve replacement (TAVR) has become an accepted treatment for inoperable patients with symptomatic severe aortic stenosis. Transcatheter aortic valve replacement repairs an aortic valve without replacing the existing damaged aortic valve, and instead wedges a replacement valve into the aortic valve's place. The replacement valve is delivered to the site through a catheter and then expanded, and the old valve leaflets are pushed out of the way. TAVR is a minimally invasive procedure in which the chest is surgically opened in (only) one or more very small incisions that leave the chest bones in place. The incision(s) in the chest can be used to enter the heart through a large artery or through the tip of the left ventricle. TAVR procedures are usually performed under fluoroscopic X-ray and transesophageal echocardiography (TEE) guidance. The fluoroscopic X-ray provides high-contrast visualization of catheter-like devices, whereas TEE shows anatomy of the heart at both high resolution and framerate. Moreover, TEE can be fused with X-ray images using known methods.

Recent trends towards echo-free TAVR procedures are mainly stimulated by the high cost of general anesthesia. General anesthesia is highly recommended for TEE-guided procedures with the aim of reducing patient discomfort. On the other hand, transthoracic echocardiography (TTE) is an external ultrasound imaging modality that may be performed without general anesthesia, using for instance conscious sedation, thus leading to shorter patient recovery times. Some disadvantages of using TTE as an intraprocedural tool in minimally invasive procedures may include:
requirements for significant experience and expertise of the imager due to high dependence on patient anatomy
non-continuous imaging due to a higher risk of radiation exposure for the sonographer compared to TEE
frequent removal of the ultrasound transducer can cause significant delays in the interventional procedure
a limited window for imaging
lack of intraoperative methods for fusing ultrasound images with X-ray fluoroscopic images (registration is available for TEE but not TTE)

As described herein, real-time tracking for fusing ultrasound imagery and x-ray imagery enables radiation-free ultrasound probe tracking so that ultrasound imagery can be overlaid onto two-dimensional and three-dimensional X-ray images.

### SUMMARY

According to an aspect of the present disclosure, a registration system includes a controller. The controller includes a memory that stores instructions, and a processor that executes the instructions. When executed by the processor, the instructions cause the controller to execute a process that includes obtaining a fluoroscopic X-ray image from an X-ray imaging system, and a visual image of a hybrid marker affixed to the X-ray imaging system from a camera system separate from the X-ray imaging system. The process also includes estimating a transformation between the hybrid marker and the X-ray imaging system, based on the fluoroscopic X-ray image, and estimating a transformation between the hybrid marker and the camera system based on the visual image. The process further includes registering ultrasound images from an ultrasound system to the fluoroscopic X-ray image from the X-ray imaging system based on the transformation estimated between the hybrid marker and the X-ray imaging system, so as to provide a fusion of the ultrasound images to the fluoroscopic X-ray image.

According to another aspect of the present disclosure, a registration system includes a hybrid marker, a camera system and a controller. The hybrid marker is affixed to an X-ray imaging system. The camera system is separate from the X-ray imaging system and has a line of sight to the hybrid marker that is maintained during a procedure. The controller includes a memory that stores instructions and a processor that executes the instructions. When executed by the processor, the instructions cause the controller to execute a process that includes obtaining a fluoroscopic X-ray image from the X-ray imaging system, and a visual image of the hybrid marker affixed to the X-ray imaging system from the camera system. The process also includes estimating a transformation between the hybrid marker and the X-ray imaging system, based on the fluoroscopic X-ray image and the visual image, and estimating a transformation between the hybrid marker and the camera system based on the visual image. The process further includes registering ultrasound images from an ultrasound system to the fluoroscopic X-ray image from the X-ray imaging system based on the transformation estimated between the hybrid marker and the X-ray imaging system.

According to yet another aspect of the present disclosure, a method of registering imagery includes obtaining, from an X-ray imaging system a fluoroscopic X-ray image; and obtaining, from a camera system separate from the X-ray imaging system, a visual image of a hybrid marker affixed to the X-ray imaging system. The method also includes estimating a transformation between the hybrid marker and the X-ray imaging system, based on the fluoroscopic X-ray image, and estimating a transformation between the hybrid marker and the camera system based on the visual image. The method further includes registering ultrasound images from an ultrasound system to the fluoroscopic X-ray image from the X-ray imaging system based on the transformation estimated between the hybrid marker and the X-ray imaging system.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1. illustrates a fusion system for real-time tracking for fusing ultrasound imagery and x-ray imagery, in accordance with a representative embodiment.

FIG. 2A illustrates an arrangement in which an ultrasound probe with an attached optical camera is positioned on an anthropomorphic torso phantom under a flat panel detector, in accordance with a representative embodiment.

FIG. 2B illustrates an optical camera integrated with an ultrasound transducer, in accordance with a representative embodiment.

FIG. 3A illustrates a hybrid marker integrated into a universal sterile drape for flat panel detectors, in accordance with a representative embodiment.

FIG. 3B illustrates a process for attaching a hybrid marker to a detector using self-adhesive tape, in accordance with a representative embodiment.

FIG. 4 illustrates a general computer system, on which a method of real-time tracking for fusing ultrasound imagery and x-ray imagery can be implemented, in accordance with a representative embodiment.

FIG. 5A illustrates radio-opaque landmarks embedded in the body of a hybrid marker, in accordance with a representative embodiment.

FIG. 5B illustrates a surface of a hybrid marker with a set of distinguishable visual features that uniquely define the coordinate system of the hybrid marker, in accordance with a representative embodiment.

FIG. 6A illustrates a process for real-time tracking for fusing ultrasound imagery and x-ray imagery, in accordance with a representative embodiment.

FIG. 6B illustrates a process for attaching a hybrid marker to a detector casing for real-time tracking for fusing ultrasound imagery and x-ray imagery, in accordance with a representative embodiment.

FIG. 6C illustrates a process for acquiring a two-dimensional fluoroscopic image for real-time tracking for fusing ultrasound imagery and x-ray imagery, in accordance with a representative embodiment.

FIG. 6D illustrates a process for positioning an ultrasound probe with integrated camera within a clinical site for real-time tracking for fusing ultrasound imagery and x-ray imagery, in accordance with a representative embodiment.

FIG. 6E illustrates a process for tracking a hybrid marker and overlaying an ultrasound image plane on the two-dimensional fluoroscopic image or the volumetric computer-tomography (CT) image for real-time tracking for fusing ultrasound imagery and x-ray imagery, in accordance with a representative embodiment.

FIG. 7A illustrates a visualization in which an ultrasound image plane is overlaid on a two-dimensional fluoroscopic X-ray image, in accordance with a representative embodiment.

FIG. 7B illustrates a visualization in which an ultrasound image plane is overlaid on a volumetric cone-beam computer-tomography image, in accordance with a representative embodiment.

FIG. 8 illustrates another process for real-time tracking for fusing ultrasound imagery and x-ray imagery, in accordance with a representative embodiment.

### DETAILED DESCRIPTION

In the following detailed description, for purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In view of the foregoing, the present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

As described below, real-time tracking for fusing ultrasound imagery and x-ray imagery uses a visual sensing component and a hybrid marker that may be attached to an X-ray imaging system detector such as a mobile C-arm flat panel detector. Real-time tracking for fusing ultrasound imagery and x-ray imagery can be implemented without requiring additional tracking hardware such as optical or electromagnetic tracking technology and is therefore readily integrated into existing clinical procedures. An example of the visual sensing component is a low-cost optical camera.

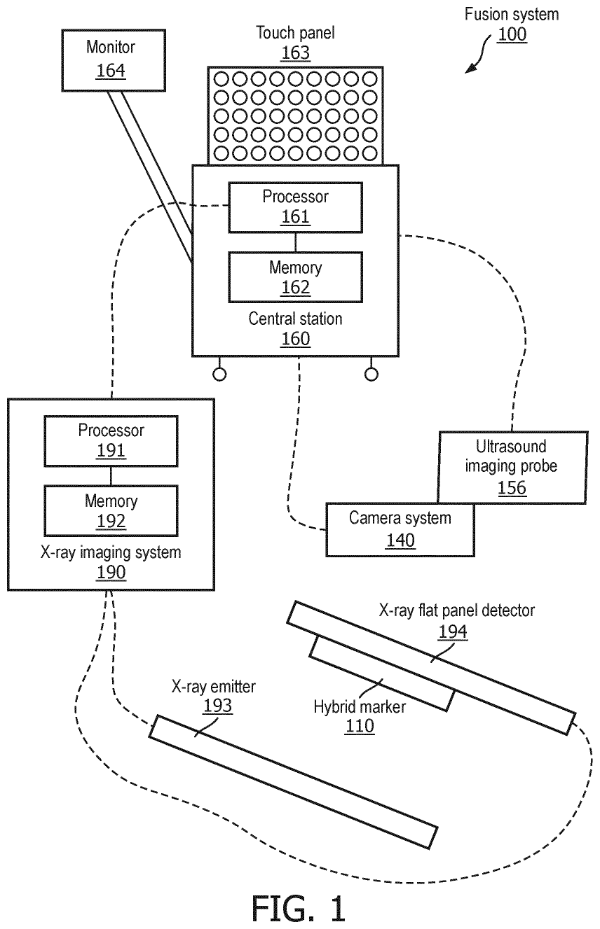

FIG. 1. illustrates a fusion system for real-time tracking for fusing ultrasound imagery and x-ray imagery, in accordance with a representative embodiment.

In the fusion system 100 of FIG. 1, an X-ray imaging system 190 includes a memory 192 that stores instructions and a processor 191 that executes the instructions. The X-ray imaging system 190 also includes an X-ray emitter 193 and an X-ray flat panel detector 194. The processor 191 executes instructions to control the X-ray emitter 193 to emit X-rays, and to control the X-ray flat panel detector 194 to detect the X-rays. A hybrid marker 110 is attached to the X-ray flat panel detector 194.

An example of the X-ray imaging system 190 is a detector-based cone beam computer-tomography imaging system such as a flat-panel detector C-arm computer-tomography imaging system. A detector-based cone beam computer-tomography imaging system may have a mechanically fixed center of rotation known as an isocenter. The X-ray imaging system 190 is configured to acquire two-dimensional fluoroscopic X-ray images, acquire volumetric cone-beam computer-tomography images, and register two-dimensional fluoroscopic X-ray images with a three-dimensional volumetric dataset using information provided by the C-arm encoders. The volumetric cone-beam computer-tomography images are an example of three-dimensional volumetric computer-tomography images that can be used in the registering described herein.

The hybrid marker 110 may be placed on the X-ray imaging system 190, and registration may be performed with the hybrid marker 110 on the X-ray imaging system 190. The hybrid marker 110 has hybrid characteristics in that the hybrid marker 110 appears both visually to the naked eye and in X-ray imagery. That is, the hybrid marker 110 is translucent to X-rays from the X-ray emitter 193 whereas a radio-opaque pattern 111 engraved in the hybrid marker 110 may appear in the imagery from the X-ray imaging system 190.

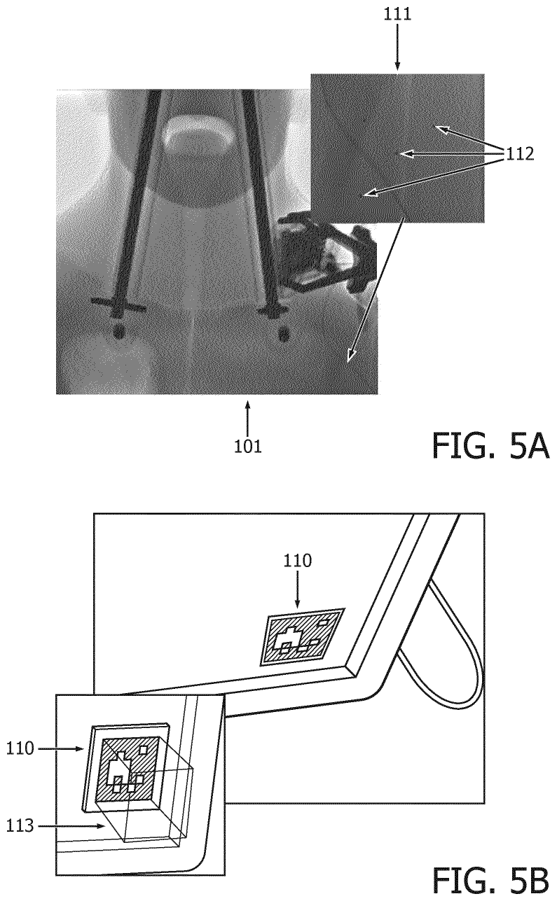

The hybrid marker 110 may be made of a material that is invisible or substantially invisible to X-rays from the X-ray emitter 193. An example of the hybrid marker 110 is a self-adhesive hybrid marker made of a plastic tape. Alternatively, a self-adhesive hybrid marker may include one surface that is part of a system of loops and hooks, or may be coated with glue. The hybrid marker 110 may also be a set of multiple markers and is integrated into a universal sterile C-arm detector drape (see FIG. 5A). The hybrid marker 110 may also comprise plastic, paper, or even metal. For example, the hybrid marker 110 may be made of paper and affixed to the X-ray imaging system 190 with tape. The hybrid marker 110 may be printed, laser cut, laser etched, assembled from multiple (i.e., different) materials.

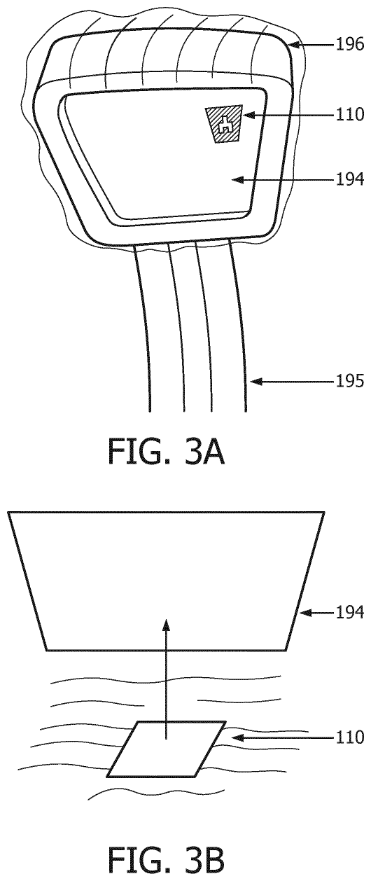

The hybrid marker 110 includes radio-opaque landmarks 112 integrated into (i.e., internalized into) a body of the hybrid marker 110 (see FIGS. 3A-3B and 5A-5B) as a radio-opaque pattern 111. Accordingly, the hybrid marker 110 may be made of a rigid or semi-rigid material such as a plastic and may have a radio-opaque pattern 111 laser-engraved onto the rigid or semi-rigid material. As an example, the hybrid marker 110 may be made of a black plastic, and the radio-opaque pattern 111 may be white so that it is easy to visually detect. When the hybrid marker 110 is made of plastic tape, the radio-opaque pattern 111 may be laser-engraved into the plastic tape, and a surface of the plastic tape may be a self-adhesive surface. The radio-opaque pattern 111 may be identical both to the naked eye and in an X-ray may image, but the pattern may also be different in the different modes so long as the relationship between the patterns is known.

The hybrid marker 110 therefore includes an external surface with the radio-opaque pattern 111 as a set of visual features (see FIG. 5B) that uniquely define a coordinate system 113 of the hybrid marker 110. The unique features of the coordinate system 113 may be asymmetric, may include dissimilar shapes, and may be arranged so that distances between different shapes of the radio-opaque pattern 111 are known in advance so that the asymmetry can be sought and recognized in image analysis in order to determine the orientation of the hybrid marker 110. In an embodiment, symmetrical and similar shapes can be used, so long as orientation of the hybrid marker 110 can still be identified in image analysis.

The hybrid marker 110 may be mounted to the casing of the image intensifier of the X-ray imaging system 190. As a result, the radio-opaque landmarks 112 which are internal can be observed on intra-procedural fluoroscopic X-ray images. An example of radio-opaque markers as landmarks is described in U.S. Patent Application Publication No. 2007/0276243. Additionally, a single marker may be used as the hybrid marker 110, since a single marker may be sufficient for tracking and registration. However, stability of the tracking can be improved by using multiple of the hybrid marker 110 in different parts of the C-arm device. For example, different markers can be placed on the detector casing, arm cover, etc. Additionally, a hybrid marker 110 can be pre-calibrated and thus integrated into the existing C-arm devices.

The fusion system 100 may also be referenced as a registration system. The fusion system 100 of FIG. 1 also includes a central station 160 with a memory 162 that stores instructions and a processor 161 that executes the instructions. A touch panel 163 is used to input instructions from an operator, and a monitor 164 is used to display images such as X-ray images fused with ultrasound images. The central station 160 performs data integration in FIG. 1, but in other embodiments some or all of the data integration may be performed in the cloud (i.e., by distributed computers such as at data centers). Thus, the configuration of FIG. 1 is representative of a variety of configurations that can be used to perform image processing and related functionality as described herein.

An ultrasound imaging probe 156 communicates with the central station 160 by a data connection. The camera system 140 is affixed to the ultrasound imaging probe 156, and also communicates with the central station 160 by a data connection. The ultrasound imaging probe 156 is an ultrasound imaging device configured to acquire two-dimensional and/or three-dimensional ultrasound images using a transducer.

The camera system 140 is representative of a sensing system and may be an optically calibrated monocular camera that is attached to and calibrated with the ultrasound imaging probe 156. The camera system 140 may be a monocular camera or a stereo camera (two or more lenses with separate, e.g., image sensor, for each lens) that is calibrated with the ultrasound imaging probe 156. The camera system 140 may also be a monochrome camera or a red/green/blue (RGG) camera. The camera system 140 may also be an infrared (IR) camera or a depth sensing camera. The camera system 140 is configured to be located under the C-arm device detector of the X-ray imaging system 190, acquire images of the hybrid marker 110 attached to the C-arm device detector, and provide calibration parameters such as an intrinsic camera matrix to a controller of the camera system 140.

The ultrasound imaging probe 156 may be calibrated to a coordinate system of the camera system 140 by a transformation (cameral ultrasound) using known methods. For instance, the hybrid marker 110 may be rigidly fixed to a phantom with photoacoustic fiducial markers (us_phantom) located therein. The phantom can be scanned using the ultrasound imaging probe 156 with the camera system 140 mounted thereon. A point-based rigid registration method known in the art can be used to calculate a transformation (^{us_phantom}T_{ultrasound}) between the photoacoustic fiducial markers located in the phantom and corresponding fiducials visualized on ultrasound images. Simultaneously, the camera system 140 may acquire a set of images of the hybrid marker 110 that is rigidly fixed to the ultrasound phantom. The transformation (^{marker}T_{us_phantom}) between the phantom and the hybrid marker 110 may be known in advance. Having set of corresponding ultrasound and cameras images one can estimate ultrasound-to-camera transformation (^{camera}T_{ultrasound}) using equation (1) below:

^{camera}T_{ultrasound}=^{camera}Tₘₐᵣₖₑᵣ·^{marker}T_{us_phantom}·^{us_phantom}T_{ultrasound} (1)

The fusion system 100 of FIG. 1 is representative of a system that includes different subsystems for real-time tracking for fusing ultrasound imagery and x-ray imagery. That is, the X-ray imaging system 190 is representative of an X-ray system used to perform X-ray imaging on a patient, the ultrasound imaging probe 156 is representative of an ultrasound imaging system used to perform ultrasound imaging on a patient, and the central station 160 is representative of a fusion system that processes imaging results from the X-ray imaging system 190 and the ultrasound imaging probe 156. The central station 160, or a subsystem of the central station 160 may also be referenced as a controller that includes a processor and memory. However, the functionality of any of these three systems or subsystems may be integrated, separated, or performed in numerous different ways by different arrangements within the scope of the present disclosure.

A controller for the camera system 140 may be provided together with, or separate from, a controller for registration. For example, the central station 160 may be a controller for the camera system 140 and for registration as described herein. Alternatively, the central station 160 may include the processor 161 and memory 162 as one controller for the camera system 140, and another processor/memory combination as another controller for the registration. In yet another alternative, the processor 161 and memory 162 may be a controller for one of the camera system 140 and the registration, and another controller may be provided separate from the central station 160 for the other of the camera system 140 and the registration.

In any event, a controller for the camera system 140 may be provided as a sensing system controller that is configured to receive images from the camera system 140, interpret information about calibration parameters such as intrinsic camera parameters of the camera system 140, and interpret information pertaining to the hybrid marker 110 such as a configuration of visual features that uniquely identify the geometry of the hybrid marker 110. The controller for the camera system 140 may also localize visual features of the hybrid marker 110 on the received images and reconstruct a three-dimensional pose of the hybrid marker 110 using the unique geometry of these features. The pose of the hybrid marker 110 can be reconstructed via the transformation (^{camera}Tₘₐᵣₖₑᵣ) using monocular images by solving a perspective-n-point (PnP) problem using known methods such as a random sample consensus (RANSAC) algorithm.

Additionally, whether a controller for registration is the same as the controller for the camera system 140 or different, the controller for registration is configured to receive fluoroscopic images from the X-ray flat panel detector 194, and interpret information from fluoroscopic images from the X-ray flat panel detector 194 to estimate a transformation (^{X-ray}Tₐᵣₖₑᵣ) between the hybrid marker 110 (i.e., located on the image intensifier) and the X-ray flat panel detector 194.

As noted, the fusion system 100 in FIG. 1 includes a monitor 164. Additionally, although not shown, the fusion system 100 may include a mouse, keyboard, or other input device even when the monitor 164 is touch-sensitive such that instructions can be input directly to the monitor 164. Based on the registration between the ultrasound images and the X-ray image(s), the ultrasound images can be overlaid onto the X-ray image(s) on the monitor 164 as a result of using the hybrid marker 110 in the manner described herein.

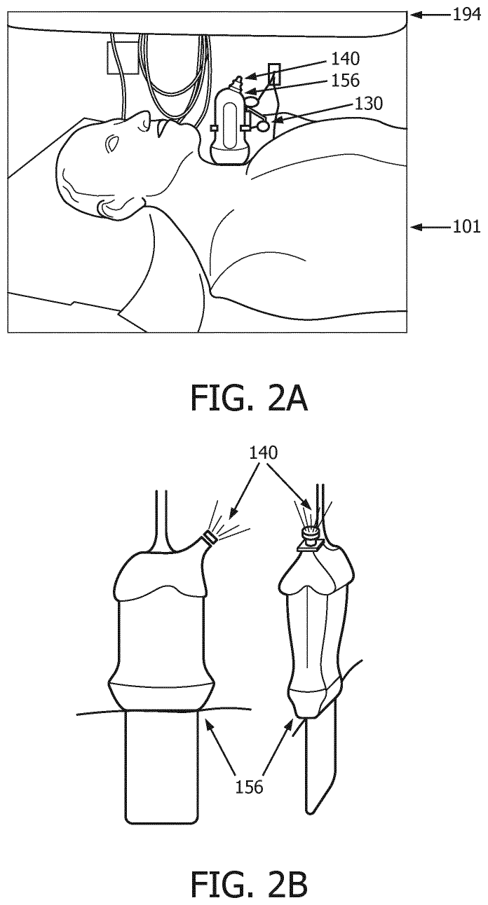

FIG. 2A illustrates an arrangement in which an ultrasound probe with an attached optical camera is positioned on an anthropomorphic torso phantom under a flat panel detector, in accordance with a representative embodiment.

In FIG. 2A, an ultrasound imaging probe 156 is shown with an attached camera system 140 and is held with an arm 130 so as to be remotely controlled or fixed in place. The ultrasound imaging probe 156 is held by the arm 130 adjacent to a neck of the anthropomorphic torso phantom 101. An X-ray flat panel detector 194 is shown above the anthropomorphic torso phantom 101.

FIG. 2B illustrates an optical camera integrated with an ultrasound transducer, in accordance with a representative embodiment.

In FIG. 2B, the camera system 140 is integrated with the ultrasound imaging probe 156, as shown in side and frontal views. The ultrasound imaging probe 156 may be referenced as an ultrasound system. The ultrasound imaging probe 156 may be manufactured with the camera system 140 integrated therein. Alternatively, the camera system 140 may be detachably affixed to the ultrasound imaging probe 156, such as with tape, glue, a fastening system with loops on one surface and hooks on another surface to hook into the loops, a mechanical clamp, and other mechanisms for detachably fixing one object to another. An orientation of the camera system 140 relative to the ultrasound imaging probe 156 may be fixed in the embodiment of FIG. 2B. However, the camera system 140 may be adjustable relative to the ultrasound imaging probe 156 in other embodiments.

FIG. 3A illustrates a hybrid marker integrated into a universal sterile drape for flat panel detectors, in accordance with a representative embodiment.

In FIG. 3A, the X-ray flat panel detector 194 is covered by a universal sterile drape 196. The X-ray flat panel detector 194 is detachably attached to a C-arm 195 that is used to perform rotational sweeps so that the X-ray flat panel detector 194 detects X-rays from an X-ray emitter 193 (not shown in FIG. 3A). A C-arm 195 is a medical imaging device and connects the X-ray emitter 193 as an X-ray source to the X-ray flat panel detector 194 as an X-ray detector. Mobile C-arms such as the C-arm 195 may use image intensifiers with a charge-coupled device (CCD) camera. Flat-panel detectors such as the X-ray flat panel detector 194 are used due to high image quality and a smaller system with a larger field of view (FOV) unaffected by geometrical and magnetic distortions.

A hybrid marker 110 is integrated into the universal sterile drape 196. When used, the hybrid marker 110 is placed into the line of sight of the camera system 140 of FIGS. 2A and 2B. The camera system 140 is mounted to the ultrasound system such as the ultrasound imaging probe 156 and maintains a line of sight to the hybrid marker 110 during a procedure.

FIG. 3B illustrates a process for attaching a hybrid marker to a detector using self-adhesive tape, in accordance with a representative embodiment.

In FIG. 3B, the hybrid marker 110 is attached to the X-ray flat panel detector 194 using self-adhesive tape.

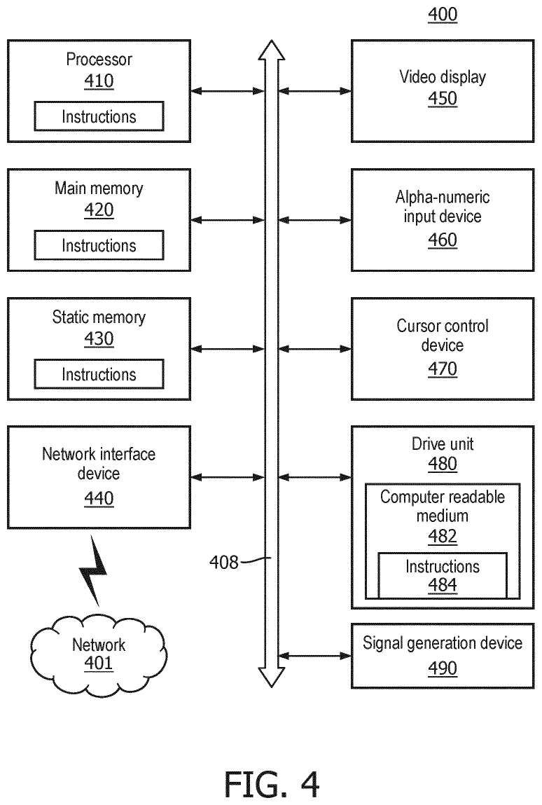

FIG. 4 illustrates a general computer system, on which a method of real-time tracking for fusing ultrasound imagery and x-ray imagery can be implemented, in accordance with a representative embodiment.

The computer system 400 can include a set of instructions that can be executed to cause the computer system 400 to perform any one or more of the methods or computer-based functions disclosed herein. The computer system 400 may operate as a standalone device or may be connected, for example, using a network 401, to other computer systems or peripheral devices. Any or all of the elements and characteristics of the computer system 400 in FIG. 4 may be representative of elements and characteristics of the central station 160, the X-ray imaging system 190, or other similar devices and systems that can include a controller and perform the processes described herein.

In a networked deployment, the computer system 400 may operate in the capacity of a client in a server-client user network environment. The computer system 400 can also be fully or partially implemented as or incorporated into various devices, such as a central station, an imaging system, an imaging probe, a stationary computer, a mobile computer, a personal computer (PC), or any other machine capable of executing a set of instructions (sequential or otherwise) that specify actions to be taken by that machine. The computer system 400 can be incorporated as or in a device that in turn is in an integrated system that includes additional devices. In an embodiment, the computer system 400 can be implemented using electronic devices that provide video or data communication. Further, while the computer system 400 is illustrated, the term “system” shall also be taken to include any collection of systems or sub-systems that individually or jointly execute a set, or multiple sets, of instructions to perform one or more computer functions.

As illustrated in FIG. 4, the computer system 400 includes a processor 410. A processor 410 for a computer system 400 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. Any processor described herein is an article of manufacture and/or a machine component. A processor for a computer system 400 is configured to execute software instructions to perform functions as described in the various embodiments herein. A processor for a computer system 400 may be a general-purpose processor or may be part of an application specific integrated circuit (ASIC). A processor for a computer system 400 may also be a microprocessor, a microcomputer, a processor chip, a controller, a microcontroller, a digital signal processor (DSP), a state machine, or a programmable logic device. A processor for a computer system 400 may also be a logical circuit, including a programmable gate array (PGA) such as a field programmable gate array (FPGA), or another type of circuit that includes discrete gate and/or transistor logic. A processor for a computer system 400 may be a central processing unit (CPU), a graphics processing unit (GPU), or both. Additionally, any processor described herein may include multiple processors, parallel processors, or both. Multiple processors may be included in, or coupled to, a single device or multiple devices.

Moreover, the computer system 400 includes a main memory 420 and a static memory 430 that can communicate with each other via a bus 408. Memories described herein are tangible storage mediums that can store data and executable instructions and are non-transitory during the time instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. A memory described herein is an article of manufacture and/or machine component. Memories described herein are computer-readable mediums from which data and executable instructions can be read by a computer. Memories as described herein may be random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. Memories may be volatile or non-volatile, secure and/or encrypted, unsecure and/or unencrypted.

As shown, the computer system 400 may further include a video display unit 450, such as a liquid crystal display (LCD), an organic light emitting diode (OLED), a flat panel display, a solid-state display, or a cathode ray tube (CRT). Additionally, the computer system 400 may include an input device 460, such as a keyboard/virtual keyboard or touch-sensitive input screen or speech input with speech recognition, and a cursor control device 470, such as a mouse or touch-sensitive input screen or pad. The computer system 400 can also include a disk drive unit 480, a signal generation device 490, such as a speaker or remote control, and a network interface device 440.

In an embodiment, as depicted in FIG. 4, the disk drive unit 480 may include a computer-readable medium 482 in which one or more sets of instructions 484, e.g. software, can be embedded. Sets of instructions 484 can be read from the computer-readable medium 482. Further, the instructions 484, when executed by a processor, can be used to perform one or more of the methods and processes as described herein. In an embodiment, the instructions 484 may reside completely, or at least partially, within the main memory 420, the static memory 430, and/or within the processor 410 during execution by the computer system 400.

In an alternative embodiment, dedicated hardware implementations, such as application-specific integrated circuits (ASICs), programmable logic arrays and other hardware components, can be constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing can be constructed to implement one or more of the methods or functionality as described herein, and a processor described herein may be used to support a virtual processing environment.

The present disclosure contemplates a computer-readable medium 482 that includes instructions 484 or receives and executes instructions 484 responsive to a propagated signal; so that a device connected to a network 401 can communicate video or data over the network 401. Further, the instructions 484 may be transmitted or received over the network 401 via the network interface device 440.

FIG. 5A illustrates radio-opaque landmarks embedded in the body of a hybrid marker, in accordance with a representative embodiment.

In the embodiment of FIG. 5A, the anthropomorphic torso phantom 101 faces out from the page and has the hybrid marker 110 on the left shoulder. The radio-opaque landmarks 112 of the radio-opaque pattern 111 are embedded in the body of the hybrid marker 110 and shown in a close-up view. As shown by the arrow, the radio-opaque landmarks 112 may be arranged in a radio-opaque pattern 111 in the body of the hybrid marker 110.

FIG. 5B illustrates a surface of a hybrid marker with a set of distinguishable visual features that uniquely define the coordinate system of the hybrid marker, in accordance with a representative embodiment.

In the embodiment of FIG. 5B, the surface of the hybrid marker 110 includes a set of radio-opaque landmarks 112 that are a radio-opaque pattern 111 of distinguishable visual features that uniquely define the coordinate system 113 of the hybrid marker 110. A coordinate system 113 of the hybrid marker 110 is projected from the hybrid marker 110 in the inset image in the bottom left corner of FIG. 5A. As shown, the hybrid marker 110 may be a rectangle with corners that can be used as part of the coordinate system 113, but also includes unique features that can be used to determine orientation of the hybrid marker 110. The unique features may be asymmetric so that asymmetry can be sought in image analysis based on an image that include the hybrid marker 110, such as by comparison with an image that includes the asymmetric pattern in the hybrid marker 110 so that the orientation of the hybrid marker 110 in use can be determined.

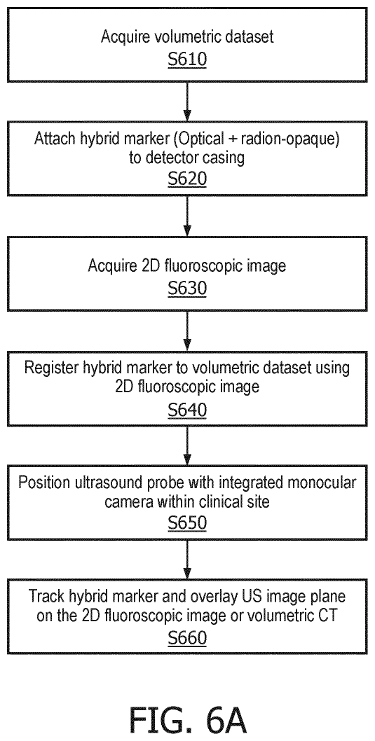

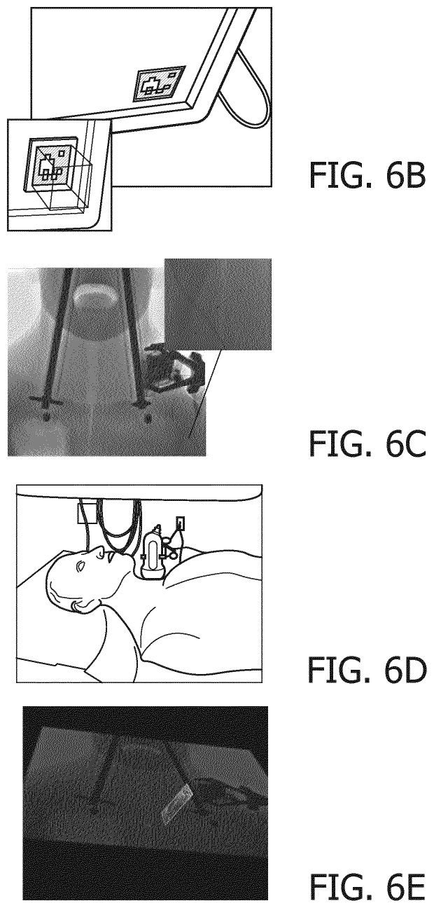

FIG. 6A illustrates a process for real-time tracking for fusing ultrasound imagery and x-ray imagery, in accordance with a representative embodiment.

In the process of FIG. 6A, a volumetric dataset is acquired at S610. The volumetric image dataset may be a computer-tomography (CT) dataset such as a cone-beam computer-tomography dataset and may be reconstructed from projections acquired from a rotational sweep of a C-arm. Alternatively, other imaging modalities can be used as soon as they can be registered to either cone-beam computer-tomography or fluoroscopic X-ray images.

A hybrid marker 110 is attached to a detector casing at S620. The hybrid marker 110 is optical plus radio-opaque. The hybrid marker 110 may be mounted to the casing of the image intensifier using self-adhesive tape. The hybrid marker 110 may be attached on the side of the detector to prevent generating streak artefacts within the volume of interest due to the radio-opaque landmarks 112 that are internal to the hybrid marker 110. To avoid streak artefacts on the computer-tomography images, the hybrid marker 110 can alternatively be fixed to the detector casing and mechanically pre-calibrated to the specific C-arm device. Alternatively, a set of at least two of the hybrid marker 110 can be used by
first, attaching both hybrid markers where a hybrid marker 110 (first hybrid marker) is positioned directly on the image intensifier (int_marker), and a hybrid marker 110 (second hybrid marker) is positioned on the external detector casing (ext_marker)
second, acquiring a pre-procedural X-ray image containing the first hybrid marker (int_marker) together with the optical camera image containing both hybrid markers, thus enabling calibration of the external marker (ext_marker) with the X-ray device, as listed by the equation (2) as follows

^{X-ray}T_{ext_marker}=^{X-ray}T_{int_marker}·(^{camera}Tᵢₙₜ_{ₘₐᵣₖₑᵣ})⁻¹·^{camera}T_{ext_marker} (2)

where both ^{camera}T_{int_marker} and ^{camera}T_{ext_marker} are provided by the sensing system controller that can estimate a three-dimensional pose of the hybrid markers, and ^{X-ray}T_{int_marker} is estimated by the registration controller
third, removing the first hybrid marker placed directly on the image intensifier (int_marker) from the C-arm for the rest of the intervention hence avoiding marker-induced image artifacts.
   In an alternative embodiment, the C-arm detector casing can contain a set of visual features that are mechanically inset and pre-calibrated (e.g., to one another) using a manufacturing process, thus providing the same functionality as previously described for the hybrid marker 110.

At S630, a two-dimensional fluoroscopic image is acquired. The two-dimensional fluoroscopic X-ray image is acquired together with the hybrid marker 110 mounted on the casing of the image intensifier, thus generating an image that is shown in FIG. 5A.

At S640, the hybrid marker 110 is registered to the volumetric dataset using a two-dimensional fluoroscopic image. For example, when the volumetric dataset is a computer-tomography dataset, the hybrid marker 110 may be registered to the computer-tomography isocenter of the volumetric dataset using the two-dimensional fluoroscopic image.

For the process at S640, a registration controller may receive a fluoroscopic X-ray image and estimate a transformation between the X-ray device and the hybrid marker 110 located on the image intensifier (^{X-ray}Tₘₐᵣₖₑᵣ). This transformation may be calculated as follows:
Assuming that the plane of the hybrid marker 110 is coplanar with the image intensifier plane, both pitch and yaw rotational components of the ^{X-ray}Tₘₐᵣₖₑᵣ transformation may be set to an identity. All manufacturing imperfections that may influence from these assumptions can be validated during the manufacturing of the X-ray device and then taken into account in this step. Similarly, one translation component (z), along the axis that is normal to the plane of the hybrid marker 110, may be set to a predetermined offset value obtained during pre-calibration process. This offset accounts for a distance between the image intensifier and the external detector casing.
Roll as well as two translational (x,y) components of the transformation may be calculated using a point-based rigid registration method as known in art, for instance one using SVD decomposition. Other rigid registration methods that may not require knowledge about corresponding point pairs, such as iterative closest point (ICP), may alternatively be used.
If required, both primary and secondary rotational angles of the C-arm are taken into account.

The calculation may also take into account certain mechanical tolerances and the static bending of the C-arm as well as suspension. All mentioned components may cause deviations of the ideal behavior and the real system pose up to several mm (0-10 mm). Usually, a two-dimensional to three-dimensional calibration is performed to take these errors into account. The result of the two-dimensional to three-dimensional calibration is stored in calibration sets that differ for various C-arm positions. A look-up table of such calibration matrixes may be used for the calculations of the ^{X-ray}Tₘₐᵣₖₑᵣ transformation.

At S650, the ultrasound probe with the integrated monocular camera is positioned within a clinical site. The ultrasound probe with the mounted optical camera is positioned under the X-ray detector in the vicinity of the clinical site. A line of sight between the camera and the hybrid marker 110 needs to be constantly provided during the procedure.

At S660, the hybrid marker 110 and overlay ultrasound image plane are tracked on the two-dimensional fluoroscopic image or a volumetric computer-tomography image. Real-time feedback for the clinician is provided using various visualization methods. Transformation for these visualization methods are calculated as follows:

^{X-ray}p=^{X-ray}Tₘₐᵣₖₑᵣ·(^{camera}Tₘₐᵣₖₑᵣ)⁻¹·^{camera}T_{ultrasound}·^{ultrasound}T_{image}·^{image}p
where ^{ultrasound}T_{image} describes mapping between image pixel space and ultrasound transducer space that accounts for pixel size and location of the image origin,
^{camera}T_{ultrasound} stands for the calibration matrix estimated using the methodology described previously, ^{camera}Tₘₐᵣₖₑᵣ is a 3D pose given by the sensing system controller, and ^{X-ray}Tₘₐᵣₖₑᵣ is estimated by the registration controller using the methodology previously described.

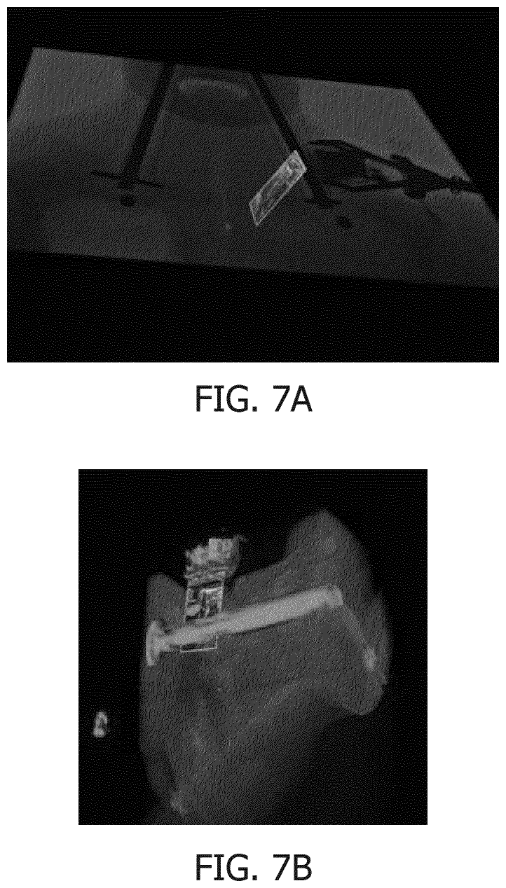

The tracking in S660 may be provided in several ways. For example, fusion of ultrasound images (including 3D ultrasound images) with fluoroscopic X-ray images is shown in FIG. 7A. Fusion of ultrasound images (including 3D ultrasound images) with volumetric cone-beam computer-tomography images is shown in FIG. 7B. Alternatively, ultrasound can be fused with other volumetric imaging modalities such as multi-slice computer-tomography, magnetic resonance imaging (MRI), and PET-CT as soon as registration between cone-beam computer-tomography and another imaging modality is provided.

Additionally, the ultrasound imaging probe 156 is described for FIG. 1 as a system external to a patient. However, a camera system 140 may be provided on or in an interventional medical device such as a needle or catheter that is used to obtain ultrasound, where the camera system 140 is provided on a portion that remains external to the patient and continuously captures the hybrid marker 110. For example, the interventional medical device may be controlled by a robotic system and may have the camera system 140 fixed thereon and controlled by the robotic system to maintain a view of the hybrid marker 110. Thus, the camera system 140 will typically always be external to the body of the patient but can be used in the context of interventional medical procedures. For example, the ultrasound imaging probe 156 may be used to monitor the angle of insertion of an interventional medical device.

In the process of FIG. 6, the fluoroscopic X-ray imagery may be obtained only once in order to acquire the volumetric dataset S610, whereas the registering of the hybrid marker 110 at S640 may be performed repeatedly. Additionally, the positioning of the ultrasound probe at S650 and the tracking of the hybrid marker 110 at S660 may be performed repeatedly or even continuously for a period, all based on the single acquisition of the volumetric dataset at S610 based on the fluoroscopic X-ray imagery. That is, a patient does not have to be repeatedly subject to X-ray imaging in the process of FIG. 6 and generally as described herein.

FIG. 6B illustrates a process for attaching a hybrid marker to a detector casing for real-time tracking for fusing ultrasound imagery and x-ray imagery, in accordance with a representative embodiment.

FIG. 6B shows the process of attaching the hybrid marker 110 to the detector casing at S620.

FIG. 6C illustrates a process for acquiring a two-dimensional fluoroscopic image for real-time tracking for fusing ultrasound imagery and x-ray imagery, in accordance with a representative embodiment.

FIG. 6C shows the process of acquiring the two-dimensional fluoroscopic image at S630.

FIG. 6D illustrates a process for positioning an ultrasound probe with integrated camera within a clinical site for real-time tracking for fusing ultrasound imagery and x-ray imagery, in accordance with a representative embodiment.

FIG. 6D shows the process of positioning the ultrasound probe with integrated monocular camera within a clinical site at S650.

FIG. 6E illustrates a process for tracking a hybrid marker and overlaying an ultrasound image plane on the two-dimensional fluoroscopic image or the volumetric computer-tomography image for real-time tracking for fusing ultrasound imagery and x-ray imagery, in accordance with a representative embodiment.

FIG. 6E shows the process of tracking the hybrid marker and overlay ultrasound image plane on the two-dimensional fluoroscopic image or volumetric computer-tomography image at S660.

FIG. 7A illustrates a visualization in which an ultrasound image plane is overlaid on a two-dimensional fluoroscopic X-ray image, in accordance with a representative embodiment.

In FIG. 7A, an ultrasound image plane is overlaid with a two-dimensional fluoroscopic X-ray image as a visualization method provided to a clinician during real-time tracking of an ultrasound probe.

FIG. 7B illustrates a visualization in which an ultrasound image plane is overlaid on a volumetric cone-beam computer-tomography image, in accordance with a representative embodiment.

In FIG. 7B, an ultrasound image plane is overlaid with a rendering of a volumetric cone-beam computer-tomography image as another visualization method provided to a clinician during real-time tracking of an ultrasound probe.

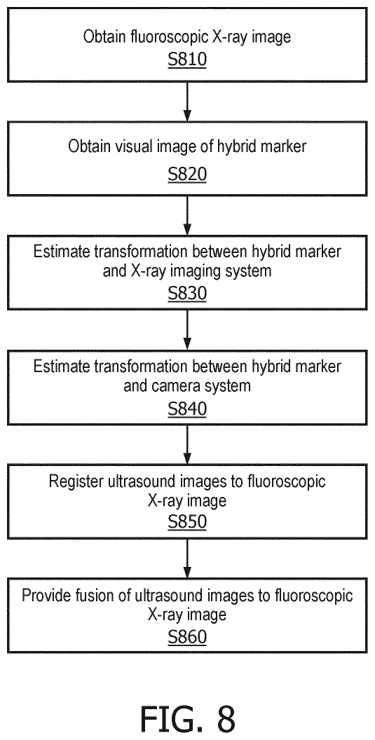

FIG. 8 illustrates another process for real-time tracking for fusing ultrasound imagery and x-ray imagery, in accordance with a representative embodiment.

In FIG. 8, the process starts at S810 with obtaining a fluoroscopic X-ray image.

At S820, a visual image of a hybrid marker 110 is obtained.

At S830, a transformation between the hybrid marker 110 and the X-ray imaging system 190 is estimated.

At S840, a transformation between the hybrid marker 110 and a camera system is estimated.

At S850, ultrasound images are registered to fluoroscopic X-ray images.

At S860, the fusion of ultrasound images to the fluoroscopic X-ray images is provided.

Accordingly, real-time tracking for fusing ultrasound imagery and x-ray imagery enables all types of image-guided procedures involving various C-arm X-ray devices ranging from low-cost mobile C-arm devices to high-end X-ray systems from hybrid operating rooms, in which usage of intra-interventional live ultrasound images could be beneficial. The image-guided procedures in which real-time tracking for fusing ultrasound imagery and x-ray imagery may be used include:
Transcatheter aortic valve replacement (TAVR)
Left atrial appendage closure (LAAO) for which usage of supplemental TTE could be beneficial,
Mitral or tricuspid valve replacement,
Other minimally-invasive procedures for structural heart diseases.

In addition, external ultrasound can be used to identify the vertebral artery increasing the safety of cervical spine procedures, including:
Cervical selective nerve root (transforaminal) injection,
Atlanto-Axial Joint Injection (pain management),
Therapeutic facet joint injection of the cervical spine,
Needle biopsy of lytic lesions of the cervical spine,
Cervical spine lesions biopsy under ultrasound,
Localization of the cervical levels,
Or other cervical spine procedures including robot-assisted cervical spinal fusion involving mobile C-arm devices.

Although real-time tracking for fusing ultrasound imagery and x-ray imagery has been described with reference to several exemplary embodiments, it is understood that the words that have been used are words of description and illustration, rather than words of limitation. Changes may be made within the purview of the appended claims, as presently stated and as amended, without departing from the scope and spirit of real-time tracking for fusing ultrasound imagery and x-ray imagery in its aspects. Although real-time tracking for fusing ultrasound imagery and x-ray imagery has been described with reference to particular means, materials and embodiments, real-time tracking for fusing ultrasound imagery and x-ray imagery is not intended to be limited to the particulars disclosed; rather real-time tracking for fusing ultrasound imagery and x-ray imagery extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

### FIG. 1

We claim:

1. A registration system (100) that includes a controller (160), the controller comprising:
a memory (162) that stores instructions; and
a processor (161) that executes the instructions,
wherein, when executed by the processor (161), the instructions cause the controller (160) to execute a process comprising:
obtaining a fluoroscopic X-ray image (S810) from an X-ray imaging system (190), and a visual image (S820) of a hybrid marker (110) affixed to the X-ray imaging system (190) from a camera system (140) separate from the X-ray imaging system (190);
estimating a transformation (S830) between the hybrid marker (110) and the X-ray imaging system (190), based on the fluoroscopic X-ray image, and estimating a transformation (S840) between the hybrid marker (110) and the camera system (140) based on the visual image; and
registering ultrasound images (S850) from an ultrasound system (156) to the fluoroscopic X-ray image from the X-ray imaging system (190) based on the transformation estimated between the hybrid marker (110) and the X-ray imaging system (190), so as to provide a fusion of the ultrasound images to the fluoroscopic X-ray image.

2. The registration system of claim 1, further comprising:
the camera system (140); and
the ultrasound system (156), wherein the camera system (140) is mounted to the ultrasound system (156) and maintains a line of sight to the hybrid marker (110) during a procedure.

3. The registration system of claim 2,
wherein the camera system (140) comprises a monocular camera or a stereo camera that is calibrated to the ultrasound system (156),
the camera system (140) provides, to the controller (160), calibration parameters defining calibration of the monocular camera or the stereo camera to the ultrasound system (156), and
the ultrasound images are registered to the fluoroscopic X-ray image based additionally on the calibration parameters.

4. The registration system of claim 1, further comprising:
the hybrid marker (110), wherein the hybrid marker (110) comprises a material translucent to X-rays from the X-ray imaging system (190) and visible in the visual image, and a radio-opaque pattern that is opaque to the X-rays from the X-ray imaging system (190).

5. The registration system of claim 4, wherein the material comprises a plastic tape, and the radio-opaque pattern in the hybrid marker (110) is engraved into the plastic tape by a laser.

6. The registration system of claim 4,
wherein the material comprises a self-adhesive surface and radio-opaque landmarks, and
the radio-opaque landmarks and the radio-opaque pattern uniquely define a coordinate system of the hybrid marker (110).

7. The registration system of claim 6, wherein the process that is executed by the controller (160) further comprises:
registering the ultrasound images from the ultrasound system (156) to the fluoroscopic X-ray image from the X-ray imaging system (190) based on capturing the radio-opaque landmarks in the fluoroscopic X-ray image from the X-ray imaging system (190).

8. The registration system of claim 1, further comprising:
the X-ray imaging system (190) from which the fluoroscopic X-ray image is received by the controller (160), wherein the X-ray imaging system (190) comprises a C-arm with an X-ray source, an image intensifier to which the hybrid marker (110) is affixed, and an encoder.

9. The registration system of claim 8, wherein the image intensifier comprises a flat-panel with a casing to which the hybrid marker (110) is affixed.

10. The registration system of claim 8, wherein the X-ray imaging system (190) is configured to perform a process comprising:
acquiring two-dimensional fluoroscopic X-ray images;
acquiring three-dimensional volumetric computer-tomography image; and
register the two-dimensional fluoroscopic X-ray images with the three-dimensional volumetric computer-tomography image.

11. The registration system of claim 8, wherein the hybrid marker (110) is integrated into the C-arm, and
the hybrid marker (110) is pre-calibrated with the C-arm prior to capturing the fluoroscopic X-ray image.

12. The registration system of claim 8, further comprising:
the camera system (140); and
the ultrasound system (156),
wherein the camera system (140) is mounted to the ultrasound system (156) and maintains a line of sight to the hybrid marker (110) during a procedure,
the camera system (140) is calibrated to the ultrasound system (156),
the camera system (140) provides, to the controller (160), calibration parameters defining calibration of the camera system (140) to the ultrasound system (156), and
the ultrasound images are registered to the fluoroscopic X-ray image based additionally on the calibration parameters.

13. A registration system (100), comprising
a hybrid marker (110) affixed to an X-ray imaging system (190);
a camera system (140), separate from the X-ray imaging system (190), and with a line of sight to the hybrid marker (110) that is maintained during a procedure; and
a controller (S160) comprising a memory (162) that stores instructions, and a processor (161) that executes the instructions,
wherein, when executed by the processor (161), the instructions cause the controller (160) to execute a process comprising:
obtaining a fluoroscopic X-ray image (S810) from the X-ray imaging system (190), and a visual image (S820) of the hybrid marker (110) affixed to the X-ray imaging system (190) from the camera system (140);
estimating a transformation (S830) between the hybrid marker (110) and the X-ray imaging system (190), based on the fluoroscopic X-ray image and the visual image, and estimating a transformation (S840) between the hybrid marker (110) and the camera system (140) based on the visual image; and
registering ultrasound images S850) from an ultrasound system (156) to the fluoroscopic X-ray image from the X-ray imaging system (190) based on the transformation estimated between the hybrid marker (110) and the X-ray imaging system (190).

14. The registration system of claim 13, further comprising:
the ultrasound system (156), wherein the camera system (140) is mounted to the ultrasound system (156) and maintains the line of sight to the hybrid marker (110) during a procedure.

15. The registration system of claim 13,
wherein the hybrid marker (110) comprises a tape translucent to X-rays from the X-ray imaging system (190), and a pattern visible in the fluoroscopic X-ray image from the X-ray imaging system (190), and
the tape comprises a plastic tape, and the pattern in the hybrid marker (110) is engraved into the plastic tape by a laser.

16. A method of registering imagery, comprising:
obtaining (S810), from an X-ray imaging system (190) a fluoroscopic X-ray image;
obtaining (S820), from a camera system (140) separate from the X-ray imaging system (190), a visual image of a hybrid marker (110) affixed to the X-ray imaging system (190);
estimating a transformation (S830) between the hybrid marker (110) and the X-ray imaging system (190), based on the fluoroscopic X-ray image and the visual image, and estimating a transformation (S840) between the hybrid marker (110) and the camera system (140) based on the visual image; and
registering (S850) ultrasound images from an ultrasound system (156) to the fluoroscopic X-ray image from the X-ray imaging system (190) based on the transformation estimated between the hybrid marker (110) and the X-ray imaging system (190).
